(19) United States
(12) Patent Application Publication
(10) Pub. No.: US 2026/0151613 A1
(43) Pub. Date: Jun. 4, 2026

(54) CIRCULATORY SUPPORT SYSTEM

(71) Applicant: BOSTON SCIENTIFIC SCIMED, INC., Maple Grove, MN (US)

(72) Inventors: Michael Kaland, White Bear Lake, MN (US); Noe Mendivil Gutierrez, Maple Grove, MN (US); Jennifer Gibbs, White Bear Township, MN (US); Corydon Carlson, Stillwater, MN (US)

(73) Assignee: BOSTON SCIENTIFIC SCIMED, INC., Maple Grove, MN (US)

(21) Appl. No.: 19/405,923

(22) Filed: Dec. 2, 2025

Related U.S. Application Data
(60) Provisional application No. 63/727,356, filed on Dec. 3, 2024.

(51) Int. Cl. A61M 60/165; A61M 60/531; A61M 60/546; A61M 60/816

(52) U.S. Cl. CPC A61M 60/165; A61M 60/531; A61M 60/546; A61M 60/816; A61M 2205/18; A61M 2205/3303; A61M 2205/3327; A61M 2205/3331; A61M 2205/3365; A61M 2205/50; A61M 2210/125; A61M 2230/005; A61M 2230/30

(57) ABSTRACT

Example medical devices, medical systems and methods of using medical systems are disclosed. An example method for monitoring the heart includes advancing a cardiac pump adjacent to a left ventricle of the heart, sensing a first parameter from a first sensor coupled to the cardiac pump, sensing a second parameter from a second sensor coupled to the cardiac pump, comparing the first parameter to a first threshold value, comparing the second parameter to a second threshold value and increasing an output of blood out of the cardiac pump based on the comparison of the first parameter to the first threshold value and the comparison of the second parameter to the second threshold value.

### CROSS REFERENCE TO RELATED APPLICATIONS

The present application claims the benefit of U.S. Provisional Patent Application Ser. No. 63/727,356, filed Dec. 3, 2024, the disclosure of which is incorporated herein by reference.

### TECHNICAL FIELD

The present disclosure relates to percutaneous circulatory support device systems. More specifically, the disclosure relates to percutaneous circulatory support devices that include heart monitoring capabilities.

### BACKGROUND

Percutaneous circulatory support devices such as cardiac blood pumps can provide transient cardiac support in patients whose heart function or cardiac output is compromised. Such devices may be delivered percutaneously from the femoral artery, retrograde through the descending aorta, over the aortic arch, through the ascending aorta across the aortic valve, and into the left ventricle. Some percutaneous circulatory support devices may include one or more sensors positioned thereon for directly measuring one or more cardiac parameters. For example, some percutaneous circulatory support devices may include sensors which derive the position of the support device within the heart, pressure levels within the heart or other cardiac parameters. In some instances, these sensors may be configured to monitor and detect dysfunction of the heart, including stoppage of the heart. Further, after detecting dysfunction or stoppage of the heart, the sensors may be configured to send one or more signals to the circulatory support device to increase the speed of the pump of the circulatory support device. Circulatory support device systems including one or more sensors configured to monitor and detect dysfunction of the heart are disclosed herein.

### SUMMARY

This disclosure provides design, material, manufacturing method, and use alternatives for medical devices and/or systems. An example method for monitoring the heart includes advancing a cardiac pump adjacent to a left ventricle of the heart, sensing a first parameter from a first sensor coupled to the cardiac pump, sensing a second parameter from a second sensor coupled to the cardiac pump, comparing the first parameter to a first threshold value, comparing the second parameter to a second threshold value and increasing an output of blood flow out of the cardiac pump based on the comparison of the first parameter to the first threshold value and the comparison of the second parameter to the second threshold value.

Alternatively or additionally to any of the embodiments above, wherein the first parameter is a torque of the cardiac pump.

Alternatively or additionally to any of the embodiments above, wherein the second parameter is a blood pressure gradient in the aorta.

Alternatively or additionally to any of the embodiments above, wherein the second parameter is a blood pressure gradient in the left ventricle.

Alternatively or additionally to any of the embodiments above, wherein the first parameter is a pulsatility of a motor of the cardiac pump.

Alternatively or additionally to any of the embodiments above, wherein the pulsatility corresponds to a signal selected from the group consisting of a current, a voltage, or a speed of the motor of the cardiac pump.

Alternatively or additionally to any of the embodiments above, wherein the pulsatility corresponds to a position of the cardiac pump relative to the left ventricle.

Alternatively or additionally to any of the embodiments above, wherein the first sensor is a torque sensor.

Alternatively or additionally to any of the embodiments above, wherein the second sensor is a pressure sensor.

Alternatively or additionally to any of the embodiments above, wherein the first parameter is compared to the first threshold value simultaneous with the second parameter being compared to the second threshold value.

Alternatively or additionally to any of the embodiments above, wherein speed of the cardiac pump is increased for a pre-set period of time.

Alternatively or additionally to any of the embodiments above, wherein the speed of the cardiac pump automatically decreases after the pre-set period of time expires.

Alternatively or additionally to any of the embodiments above, further comprising generating an alarm coincident with increasing the speed of the cardiac pump.

Alternatively or additionally to any of the embodiments above, wherein the first sensor is attached to an outer surface of the distal end region of the cardiac pump.

Another method for monitoring the heart includes advancing a cardiac pump adjacent to a left ventricle of the heart, sensing a first parameter from a first sensor coupled to the cardiac pump, sensing a second parameter from a second sensor coupled to the cardiac pump, comparing the first parameter to a first threshold value, comparing the second parameter to a second threshold value and automatically adjusting a flow of blood out of the cardiac pump based on the comparison of the first parameter to the first threshold value and the comparison of the second parameter to the second threshold value.

Alternatively or additionally to any of the embodiments above, wherein the first parameter is a torque of the cardiac pump.

Alternatively or additionally to any of the embodiments above, wherein the second parameter is a blood pressure gradient.

Alternatively or additionally to any of the embodiments above, wherein the first parameter is compared to the first threshold value simultaneous with the second parameter being compared to the second threshold value.

Alternatively or additionally to any of the embodiments above, wherein speed of the cardiac pump is increased for a pre-set period of time.

An example cardiac pump system includes a console including a processor and a cardiac pump in communication with the console. Further, the cardiac pump includes a motor, a first sensor and a second sensor. Further, the first sensor is configured to sense a first parameter, the second sensor is configured to sense a second parameter and the speed of the motor is configured to increase based on a comparison of the first parameter to a first threshold value and a comparison of a second parameter to a second threshold value.

An example cardiac pump system includes a console including processor and a cardiac pump in communication with the console. Further, the processor is configured to receive a first parameter sensed by a first sensor positioned along the cardiac pump, receive a second parameter sensed by a second sensor positioned along the cardiac pump, compare the first parameter to a first threshold value, compare the second parameter to a second threshold value and increase an output of blood out of the cardiac pump based on the comparison of the first parameter to the first threshold value and the comparison of the second parameter to the second threshold value.

Alternatively or additionally to any of the embodiments above, wherein the first parameter is a torque of the cardiac pump.

Alternatively or additionally to any of the embodiments above, wherein the second parameter is a blood pressure gradient in the aorta.

Alternatively or additionally to any of the embodiments above, wherein the second parameter is a blood pressure in the left ventricle.

Alternatively or additionally to any of the embodiments above, wherein the first parameter is a pulsatility of a motor of the cardiac pump.

Alternatively or additionally to any of the embodiments above, wherein the pulsatility corresponds to a signal selected from the group consisting of a current, a voltage, or a speed of the motor of the cardiac pump.

Alternatively or additionally to any of the embodiments above, wherein the pulsatility corresponds to a position of the cardiac pump relative to the left ventricle.

Alternatively or additionally to any of the embodiments above, wherein the first sensor is a pressure sensor.

Alternatively or additionally to any of the embodiments above, wherein the second sensor is a torque sensor.

Alternatively or additionally to any of the embodiments above, wherein the first parameter is compared to the first threshold value simultaneous with the second parameter being compared to the second threshold value.

Alternatively or additionally to any of the embodiments above, wherein speed of the cardiac pump is increased for a pre-set period of time.

Alternatively or additionally to any of the embodiments above, wherein the speed of the cardiac pump automatically decreases after the pre-set period of time expires.

Alternatively or additionally to any of the embodiments above, wherein the processor is further configured to generate an alarm coincident with increasing the speed of the cardiac pump.

Alternatively or additionally to any of the embodiments above, wherein the second sensor is attached to an outer surface of the distal end region of the cardiac pump.

Alternatively or additionally to any of the embodiments above, wherein the second sensor is attached to an outer surface of a catheter shaft coupled to a distal end region of the cardiac pump.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

FIG. 1 depicts a percutaneous circulatory support system, including a circulatory support device and its relative position in a heart of a patient;

FIG. 2 is a schematic block diagram of a console management system;

FIG. 3 depicts a portion of the circulatory support system shown in FIG. 1 positioned in the heart of a patient;

FIG. 4 is a schematic block diagram of a heart monitoring system;

FIG. 5 is a schematic block diagram of a portion of the heart monitoring system of FIG. 4;

FIG. 6 is a schematic block diagram of a portion of the heart monitoring system of FIG. 4;

FIG. 7 is a schematic block diagram of a heart monitoring system; and

FIG. 8 is a schematic block diagram of a heart monitoring system.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

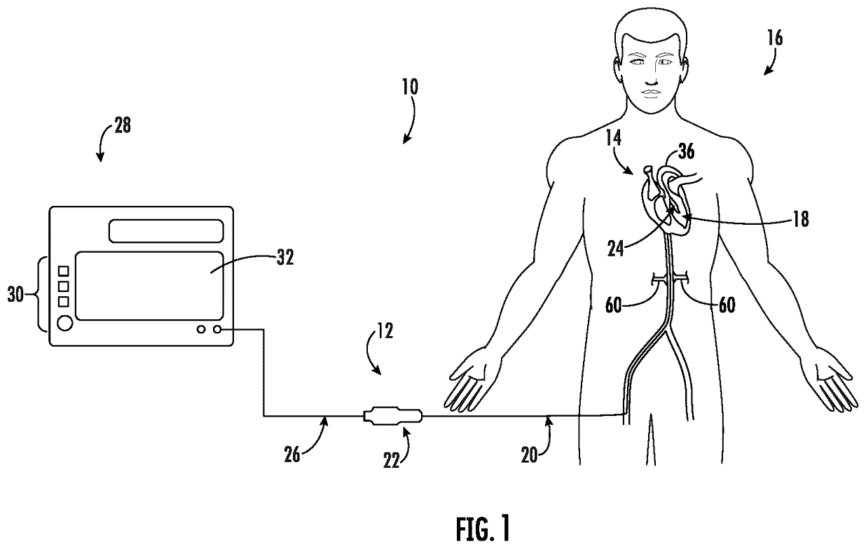

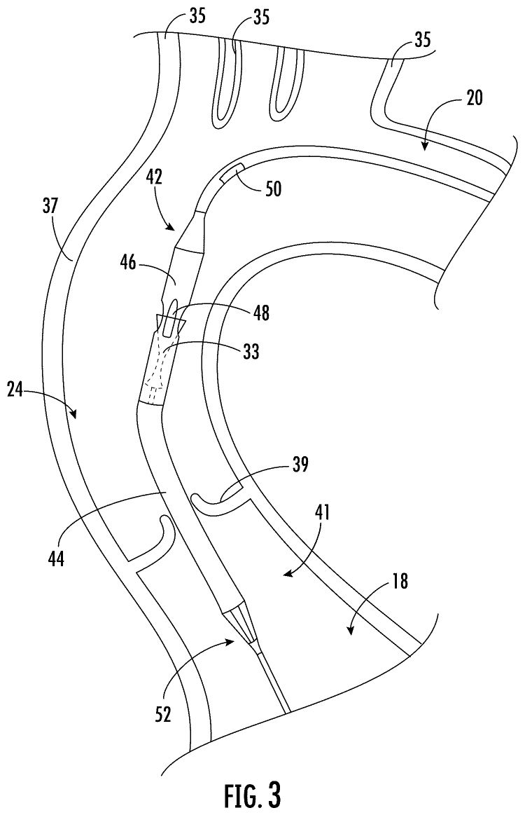

FIG. 1 illustrates an example percutaneous circulatory system 10 including a circulatory support device 12 positioned in the heart 14 of a patient 16. The circulatory support device 12 may include a flexible elongated catheter shaft 20 having a first end attached to a controller 22 and a second end attached to a blood pump 24. FIG. 1 illustrates the blood pump 24 positioned in the left ventricle 18 of the patient 16. The blood pump 24 may be delivered (e.g., tracked) to the ventricle 18 percutaneously over a guidewire. For example, the catheter shaft 20 and blood pump 24 may be tracked over a guidewire through the femoral artery, past the renal arteries 60 and the descending aorta, over the aortic arch 36, through the ascending aorta 37 (shown in FIG. 3), past the aortic valve 39 (shown in FIG. 3) and into the left ventricle 18.

FIG. 1 further illustrates that the controller 22 may include a distal end region attached to the catheter shaft 20 and a proximal end region attached to an electrical cable 26. The electrical cable 26 may include a distal end region connected to a console 28. It can be appreciated that the controller 22 may include one or more actuators (e.g., buttons, levers, dials, switches, etc.) configured to permit a clinician or other medical personnel to control various functions of the blood pump 24. For example, a clinician may be able to control the speed of the motor and/or an impeller located of the blood pump 24 via actuation of one or more actuators located on the controller 22. It can be appreciated that the speed of the motor of the blood pump 24 may correspond to the volume of blood output by the blood pump 24. Additionally, blood pump 24 operations (i.e., control of motor and/or impeller speed) that are present on the controller 22 may be integrated into the console 28 and the controller itself may be a simple connector.

Additionally, FIG. 1 illustrates that the console 28 may include one or more control knobs (e.g., buttons, knobs, dials, etc.) 30 and/or one or more displays. For example, FIG. 1 illustrates the console 28 may include a display 32. It can be appreciated that the console 28 may include more than one display. Additionally, while FIG. 1 illustrates the display 32 integrated into the console 28, it is contemplated that the circulatory system 10 may be configured such that the display 32 may be a separate, distinct component of the circulatory system 10. In other words, the first display 32 may be a separate stand-alone display, apart from the console 28. In some examples, the display 32 may get its data from a separate source.

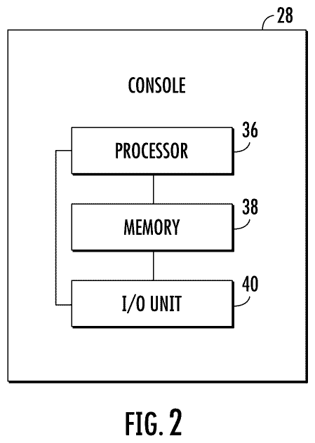

FIG. 2 illustrates that the console 28 may include, among other suitable components, one or more processors 36, memory 38, and an input/output (I/O) unit 40. The processor 36 of the console 28 may include a single processor or more than one processor working individually or with one another. The processor 36 may be configured to execute instructions, including instructions that may be loaded into the memory 38 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 36 of the console 28 may be configured to execute program instructions. Program instructions may include, for example, firmware, microcode or application code that is executed by the processor 36, a microprocessor and/or microcontroller. The one or more processors 36 may be configured to each manage different functions. They may also be configured to concurrently perform the same functions (e.g., redundant system). Further yet, they may be configured such that a first processor 36 performs a given function and second processor 36 checks the result of the function of the first processor 36 for correctness (e.g., command-monitor system).

In some examples, the display 32 may be controlled primarily by the console's firmware control instructions and, therefore, may require relatively little processing power, relatively few instructions and very simple communication between the processor 36 and the display 32. In other examples, the display 32 may be controlled primarily by an embedded computer with a flexible and relatively complex communication protocol.

The memory 38 of the console 28 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory 38 may be or may include a non-transitory computer readable medium.

The I/O units 40 of the console 28 may include a single I/O component or more than one I/O component each working individually or with one another. Example I/O units 40 may be any type of communication port configured to communicate with other components of the circulatory system 10. Example types of I/O units 45 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

FIG. 3 illustrates the blood pump 24 of the percutaneous circulatory system 10 extending from the ascending aorta 37 to the left ventricle 18 of a patient 16. The blood pump 24 may include a cannula 44 having a proximal end attached to a distal end of an impeller housing 46. A proximal end 42 of the impeller housing 46 may be attached to a distal end of the catheter shaft 20. FIG. 3 illustrates that, in some examples, the blood pump 24 may be positioned within the heart 14 such that the cannula 44 passes through the aortic valve 39, whereby a distal end region 41 of the cannula 44 may be positioned within the left ventricle 18. As discussed herein, the blood pump 24 may be tracked over a guidewire to its position illustrated in FIG. 3.

FIG. 3 further illustrates that the shaft 20 of the circulatory support device 12 may include one or more blood inlets 52 located on a distal end region 41 of the cannula 44, and one or more blood outlets 48 positioned along the impeller housing 46. In some examples, the blood pump 24 may be positioned within the heart 14 such that the one or more blood inlets 52 positioned along the distal end region 41 of the cannula 44 may be positioned in the left ventricle 18 and the one or more blood outlets 48 located along the impeller housing 46 may be positioned in the ascending aorta 37.

Additionally, the blood pump 24 may include an electrically powered motor that drives rotation of the impeller 33 which may be positioned within the impeller housing 46. In some examples, the motor may power the rotation of the impeller 33 via electromagnetic induction. The spinning impeller 33 may draw blood from the left ventricle 18 (via the one or more blood inlets 52 located on a distal region of the cannula 44) into the ascending aorta 37 (via the one or more blood outlets 48 located along the impeller housing 46). In other words, an electrically powered motor drives the impeller 33 to pump blood from the left ventricle 18 through the aortic valve 39 and into the ascending aorta 37.

Additionally, the circulatory support device 12 may include one or more sensors coupled to the cannula 44, the impeller housing 46 and/or the catheter shaft 20. The one or more sensors coupled to the cannula 44, the impeller housing 46 and/or the catheter shaft 20 may be configured to monitor blood pressures (e.g., arterial pressure, venous pressure) or other relevant cardiac parameters. Additionally, the one or more sensors of the circulatory support device 12 coupled to the cannula 44, the impeller housing 46 and/or the catheter shaft 20 may be configured to monitor other parameters of the circulatory system 10, the circulatory support device 12 and/or the patient 16. For example, the one or more sensors of the circulatory support device 12 coupled to the cannula 44, the impeller housing 46 and/or the catheter shaft 20 may be configured to monitor pulsatility in the motor torque, whereby the pulsatility in the motor torque may correspond to the position, or a change in position, of the blood pump 24.

FIG. 3 illustrates that, in some examples, the percutaneous circulatory system 10 may include a sensor 50 (e.g., pressure sensor) positioned along a distal end region of the catheter shaft 20. In other examples, the pressure sensor 50 may be positioned along the cannula 44 or the impeller housing 46. The pressure sensor 50 may be configured to sense the pressure of the blood within an ascending aorta 37 at a position adjacent to the aortic valve 39. For example, the pressure sensor 50 may be configured to directly measure the pressure of blood passing from the left ventricle 18, through the aortic valve 39 and into the ascending aorta 37. The example pressure sensor 50 described herein may include electrical sensors (e.g., MEMS sensor, membrane sensor, etc.), optical sensors (e.g., intensity sensor, interferometry sensor, etc.) or similar sensors.

Further, in some examples, the percutaneous circulatory system 10 may utilize the motor as a torque sensor by measuring its voltage, speed, and/or current. In some instances, the console may be configured to decode voltage, speed, and/or current information sensed by the motor. As will be discussed in greater detail below, in some instances the position of the blood pump 24 may be determined by comparing a pulsatility measurement taken by the torque or pressure sensors with pre-determined pulsatility values which correspond to the position of the blood pump 24 within the heart. For example, the processing components 36 of the percutaneous circulatory system 10 may include an algorithm configured to receive and compare the pulsatility data sent from the torque or pressure sensors with the pre-determined pulsatility values which correspond to the position of the blood pump 24 within the heart

It can be appreciated that any of the sensors described herein (e.g., pressure sensor 50, torque sensors, etc.) may send signals to the console 28 and/or the processing components 36 via a wireless connection (e.g., a Bluetooth connection). In other examples, any of the sensors described herein (e.g., pressure sensor 50, position sensors, etc.) may be hardwired to the console 28 and/or the processing components 36 using optical or electrical connections.

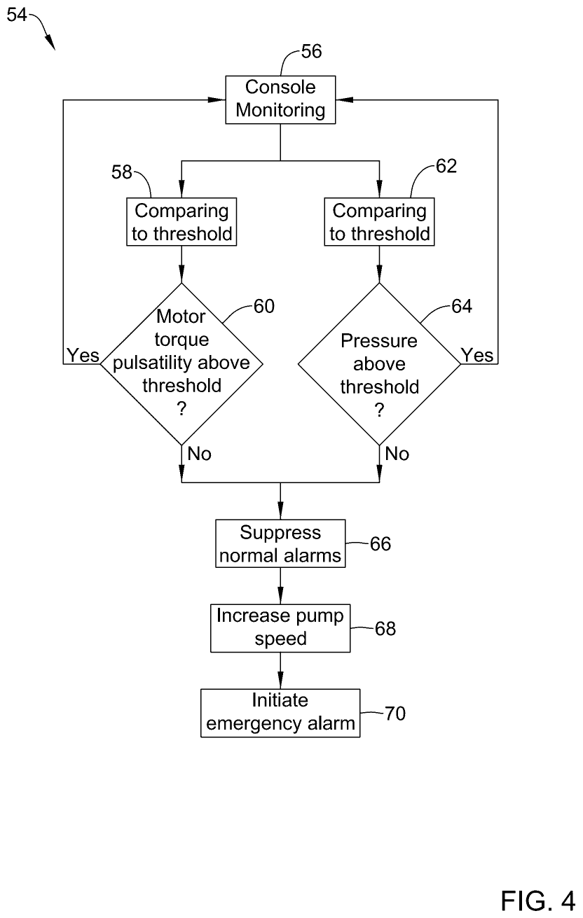

In some examples, it can be appreciated that the processing components 36 of the system 10 may be configured to receive one or more signals from the pressure sensor 50 and/or the torque sensor, process and compare those signals to pre-determined signal thresholds and change the operation of the blood pump 24 in response to the signals received and processed from the pressure sensor 50 and/or the torque sensor. For example, the processing components 36 of the system 10 may be configured to provide automatic emergency support in response to the stoppage of a patient's heart without the intervention of a physician or other operator. FIGS. 4-6 illustrate a methodology whereby the system 10 may provide automatic emergency support in response to the sudden stoppage of a patient's heart without the intervention of a physician or other operator.

FIG. 4 is a schematic diagram of an illustrative method 54 for providing automatic emergency support in response to the sudden stoppage of a patient's heart. FIG. 4 illustrates a console monitoring system 56, which may be similar in form and function to the console 28 including, among other suitable components, one or more processors 36, memory 38, and an I/O unit 40 as described herein. As discussed herein, it can be appreciated that the console 28 may be in communication with the pressure sensor 50 and/or the torque sensor. For example, the console 28 may be configured to receive one or more signals from the pressure sensor 50 and/or the torque sensor, process and compare 62, 58 those signals to pre-determined signal thresholds and change the operation of the blood pump 24 in response to the signals received and processed from the pressure sensor 50 and/or the torque sensor.

FIG. 4 further illustrates that the console may be configured to receive a signal from the torque sensor, whereby the signal received from the torque sensor may include information (e.g., data) corresponding to the pulsatility in the torque of the blood pump 24 motor. It can be appreciated that the pulsatility of the torque may be high if the pump is in a proper position. If the pulsatility of the torque is low, the pump may be out of position or the patient's heart may be beating improperly or not beating at all. Further, if the torque sensor loses pulsatility the torque sensor may be experiencing a failure or it may indicate that the patient's heart may be beating improperly or not beating at all. Additionally, it can be appreciated that the processing components 36 of the system 10 may be configured to process a signal received from the torque sensor and compare the processed signal (e.g., pulsatility signal) to a predetermined threshold. The predetermined threshold may include a pre-programmed data threshold (e.g., pulsatility level) which corresponds to a position of the blood pump 24 within the heart. It can be further appreciated that the pre-programmed data threshold (e.g., pulsatility level) may correspond to a shifting of the position of the blood pump 24. In some examples, the shifting of the position of the blood pump 24 may provide an indication that the patient's heart has suddenly stopped. Accordingly, the processing components 36 of the console 28 may determine that a patient's heart has suddenly stopped via monitoring the pulsatility of the motor torque (which, as described herein, may correspond to the position of the blood pump).

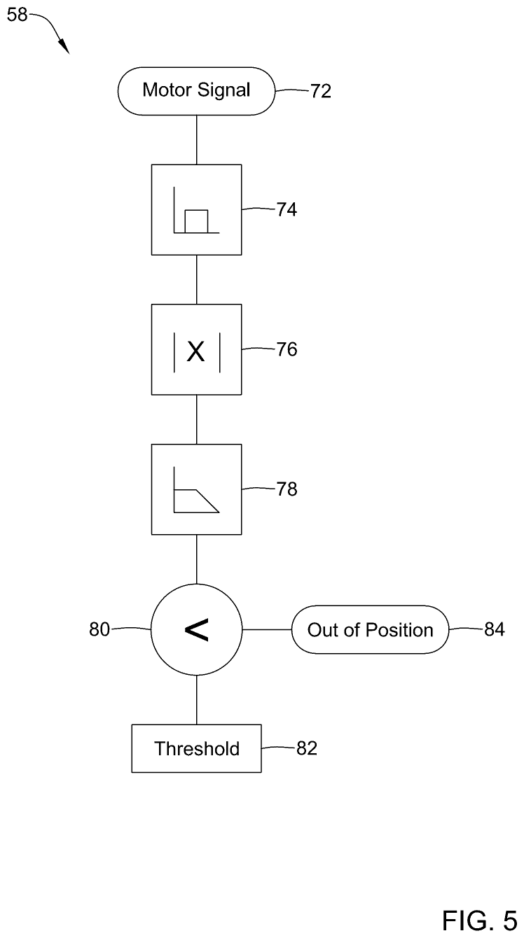

FIG. 5 illustrates that an example step in the method for providing automatic emergency support in response to the sudden stoppage of a patient's heart may include comparing a processed signal (e.g., pulsatility signal) to a predetermined threshold. A comparing step 58 may include the processing of the pulsatility signal. As described herein, the signal received from the torque sensor may correspond to the pulsatility in the torque of the blood pump 24 motor. FIG. 5 illustrates the motor torque signal 72 may be passed through a bandpass filter 74. The bandpass filter 74 may select and isolate a specific band of frequencies corresponding to the pulsatility of the torque of the motor while rejecting other frequencies. For example, a frequency range of 0.1 to 3.0 Hz may be isolated with the bandpass filter 74. Further, FIG. 5 illustrates that an envelope 76 of the motor torque signal 72 (after it has been passed through the band pass filter 74) may be taken. Further, after taking the envelope 76 of the motor torque signal, the signal may be passed through a lowpass filter 78. For example, a 0.05 Hz lowpass filter may be utilized to process the motor torque signal. As discussed herein, after passing through the lowpass filter 78, the motor torque signal may be compared 80 to a pre-programmed threshold value 82. If the pulsatility of the signal drops below the pre-programmed data threshold 82, the blood pump 24 may be determined to be out of position 84 and that the patient's heart has suddenly stopped. In some examples, a threshold of about 0.06 Newton-meter per second (Nm/s) to about 6.0 Nm/s, about 0.1 Nm/s to about 3 Nm/s, about 0.1 Nm/s to about 1.0 Nm/s, or about 0.6 Nm/s, may be utilized as the pre-programmed data threshold 82. FIG. 4 further illustrates that if the processing components 36 of the system 10 determine 60 that the pulsatility of the signal continues to exceed the pre-programmed data threshold, the blood pump 24 will continue operating normally.

FIG. 4 further illustrates that the console may be configured to receive a signal from the pressure sensor 50, whereby the signal received from the pressure sensor 50 may include information (e.g., data) corresponding to the blood pressure in the aorta of a patient. Additionally, it can be appreciated that the processing components 36 of the system 10 may be configured to process a signal received from the pressure sensor 50 and compare the processed signal (e.g., pressure signal gradient or rate of change) to a predetermined threshold. The predetermined threshold may include a pre-programmed data threshold (e.g., pressure level) which may correspond to a state of dysfunctioning heart. It can be further appreciated that the pre-programmed data threshold (e.g., pressure level) may provide an indication that the patient's heart has suddenly stopped. For example, a non-zero pressure gradient signal may indicate that the pressure sensor is functioning, while a pressure gradient signal of zero pressure may indicate that a patient's heart has suddenly stopped. Accordingly, the processing components 36 of the console 28 may determine that a patient's heart has suddenly stopped via monitoring the pressure signal received from the pressure sensor 50.

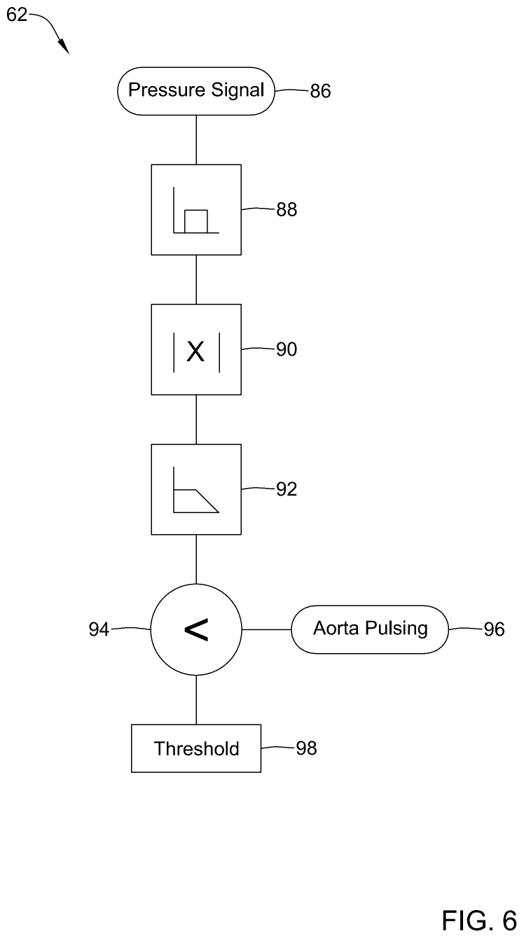

FIG. 6 illustrates that an example step in the method for providing automatic emergency support in response to the sudden stoppage of a patient's heart may include comparing a processed signal (e.g., pressure signal) to a predetermined threshold. A comparing step 62 may include the processing of the pressure signal. As described herein, the signal received from the pressure sensor 50 may correspond to the blood pressure in the aorta or ventricle of a patient. FIG. 6 illustrates that the aorta pressure or ventricle signal 86 may be passed through a band pass filter 88. For example, a frequency range of 0.1 to 3.0 Hz may be isolated with the bandpass filter 88. The bandpass filter 88 may select and isolate a specific band of frequencies corresponding to the blood pressure in the aorta or ventricle of a patient while rejecting other frequencies. Further, FIG. 6 illustrates that an envelope 90 of the pressure signal 86 (after it has been passed through the bandpass filter 88) may be taken. Further, after taking the envelope 90 of the pressure signal, the signal may be passed through a low pass filter 92. For example, a 0.05 Hz lowpass filter may be utilized to process the pressure signal. As discussed herein, after passing through the lowpass filter 92, the pressure signal may be compared 94 to a pre-programmed threshold value 98. If the pressure of the signal drops below the pre-programmed data threshold 98, it may be determined that the patient's heart has suddenly stopped. In some examples, a threshold of about 50 millimeters of mercury (mmHg/s) to about 5,000 mmHg/s, about 100 mmHg/s to about 1,000 mmHg/s, or about 500 mmHg/s, may be utilized as the pre-programmed data threshold 98. FIG. 4 further illustrates that if the processing components 36 of the system 10 determine 60 that the pressure signal continues to exceed the pre-programmed data threshold, the blood pump 24 will continue operating normally.

FIG. 4 further illustrates that if the processing components 36 of the system 10 determine 60 that the pulsatility of the motor torque signal is below the pre-programmed threshold 82 for pulsatility and also determine 64 that the pressure gradient of the pressure signal is below the pre-programmed threshold 98, the processing components 36 may determine that the patient's heart has suddenly stopped. Accordingly, FIG. 4 illustrates that if the processing components 36 determine that the patient's heart has suddenly stopped, the processing components 36 may send a signal to suppress 66 one or more alarms that would normally be associated with minor variations of the blood pump 24 being out-of-position and/or variations in aortic pressure.

Further, if the processing components 36 determine that the patient's heart has suddenly stopped, the processing components 36 may send a signal to the motor of the blood pump 24 to increase 68 the speed of the motor of the blood pump 24, thereby providing an increased flow of blood within the aorta. The increased pumping speed may increase the flow of blood within the aorta to levels which are out of the pre-programmed range for a normal operating condition of the blood pump 24. In other words, when the processing components 36 determine and detect a patient's heart has suddenly stopped, the processing components 36 may automatically increase the speed of the motor of the blood pump 24, thereby providing an increased boost of blood flow within the aorta. In some examples, the processing components 36 may automatically increase the speed of the motor of the blood pump 24 for a pre-set period of time. The increased flow of blood may provide medical personnel with adequate blood flow for a time period (e.g., a pre-set time period) to address the root cause of the patient's heart stoppage. It can be appreciated that if the patient's heart begins beating while the blood pump 24 is operating at an increased speed, the processing components 36 may send a signal to the blood pump 24 to reduce the speed to a normal operating condition.

Further, in some examples, the increased blood flow may continue for a pre-programmed length of time (e.g., pre-set period of time), at which point the motor may slow down to a normal operating condition after the expiration of the programmed length of time (e.g., pre-set period of time). In other words, the speed of the cardiac pump may automatically decrease after the pre-set period of time expires. In some examples, the pre-programmed length of time (e.g., pre-set period of time) may be about 1 minute to about 30 minutes. In other examples, the increased blood flow may continue until an operator manually slows the motor of the blood pump 24 to a normal operating condition via one or more actuation buttons on the console 28 and/or controller 22. Additionally, the console may also include a manual “override” actuation button which permits medical personnel to maintain the increased motor speed upon the expiration of the pre-programmed time period (e.g., pre-set period of time) for increased motor speed. In other words, if the increased motor speed is pre-programmed (e.g., pre-set) to run for a given amount of time, the console 28 may include an actuation button which, upon actuation by medical personnel, overrides the pre-programmed time limit and maintains the increased motor speed.

FIG. 4 further illustrates that if the processing components 36 determine that the patient's heart has suddenly stopped and an increase of the speed of the blood pump 24 is initiated, the processing components 36 may also initiate 70 the sounding of an emergency alarm coincident with the increase of the speed of the blood pump 24 to notify medical personnel that the system 10 is operating in an emergency and time-limited mode.

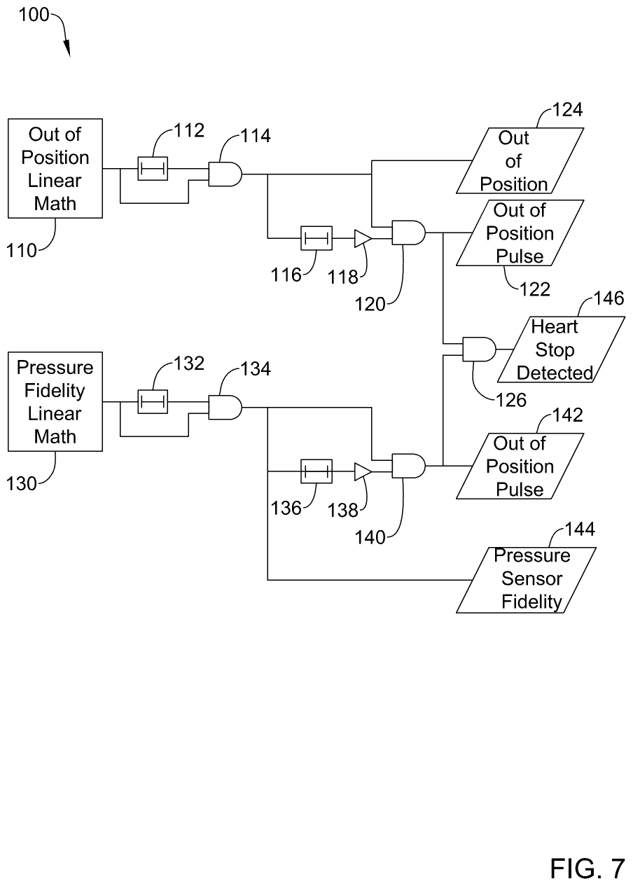

FIG. 7 is an example signal processing diagram 100 for providing automatic emergency support in response to the sudden stoppage of a patient's heart. The signal processing diagram 100 of FIG. 7 illustrates that a signal corresponding to the relative position (sensed by the torque sensor) of the blood pump 24 in the patient's heart may be processed 110 via one or more algorithms. FIG. 7 illustrates that the position signal may be sent to a time delay function 112. The time delay function 112 may delay the passing of the position signal for up to 0.5 seconds. After the position signal has passed through the time delay function 112, both the time delayed position signal and a non-time delayed position signal may pass into an “AND” logic gate 114. It can be appreciated that if both the time delayed position signal and the non-time delayed position signal inputs are “true” (e.g., both the time delayed position signal and the non-time delayed position signal indicate the blood pump is out of position), the blood pump may be out of position within the heart, which is depicted by box 124. However, if one of the input signals entering the AND logic gate 114 are false, the output signal of the AND logic gate 114 will be false, indicating that that the blood pump is not out of position. Further, this false signal may pass to a second time delay function 116. The second time delay function may delay the passing of the position signal for up to five (5) seconds. After the signal has passed through the second time delay function 116, it may pass into an inverter logic gate 118. If the second time delayed signal is false, the inverter gate 118 may invert the signal and output a true signal (indicating that the blood pump is out of position) to a second AND logic gate 120. If the signal passing from the inverter gate 118 to the second AND logic gate 120 is true, the situation may indicate that the blood pump was not out of position five (5) seconds prior to entering the second AND logic gate 120 but is out of position at the present time. If the signal passing into the second AND logic gate 120 from the inverter gate 118 and the signal passing into the second AND logic gate 120 from the first AND logic gate 114 are both true, then an out of position “pulse” may have occurred, which is depicted by box 122. The out of position pulse indicates a brief window where the system looks for indications that the pump is not actually out of position, but where the patient's heart may be functioning abnormally.

The signal processing diagram 100 of FIG. 7 further illustrates that a signal corresponding to the pressure (sensed by the pressure sensor) in the patient's heart may be processed 130 via one or more algorithms. FIG. 7 illustrates that the pressure signal may be sent to a third time delay function 132. The third time delay function 132 may delay the passing of the pressure signal for up to 0.5 seconds. After the pressure signal has passed through the third time delay function 132, both the time delayed pressure signal and a non-time delayed pressure signal may pass into a third “AND” logic gate 134. It can be appreciated that if both the time delayed position signal and the non-time delayed pressure signal inputs are “true” (e.g., both the time delayed position signal and the non-time delayed position signal indicate the pressure in the heart is below a pre-set threshold), the blood pressure sensor may be correctly determining that the pressure in the heart is abnormally low or that the pressure is static (in other words, the rate of pressure change is too low), which is indicated by box 144. However, if one of the input signals entering the third AND logic gate 134 are false, the output signal of the third AND logic gate 134 will be false, indicating that the pressure (or the pressure rate) is not abnormally low. Further, this false signal may pass to a fourth time delay function 136. The fourth time delay function 136 may delay the passing of the pressure signal for up to five (5) seconds. After the pressure signal has passed through the fourth time delay function 136, it may pass into a second inverter logic gate 138. If the time delayed signal is false, the inverter logic gate 138 may invert the signal and output a true signal (indicating that the pressure, or the pressure rate, in the heart is abnormally low) to a fourth AND logic gate 140. If the signal passing from the inverter gate 138 to the fourth AND logic gate 140 is true, the situation may indicate that the pressure or pressure rate in the heart was normal five (5) seconds prior to entering the fourth AND logic gate 140 but is low at the present time when entering the fourth AND logic gate 140. If the signal passing into the fourth AND logic gate 140 from the inverter gate 138 and the signal passing into the fourth AND logic gate 140 from the third AND logic gate 134 are both true, then a pressure monitor “pulse” may have occurred, which is depicted by box 142. The low pressure or pressure rate pulse may indicate a special window in which the system compares results against the position monitor to determine if the pump is actually in position and the heart is beating abnormally.

Further, after passing through the second AND logic gate 120, the position signal may enter a fifth AND logic gate 126. Additionally, after passing through the fourth AND logic gate 140, the pressure signal may enter fifth AND logic gate 126. If both of these signals are true, a situation in which the patient's heart has stopped may be indicated as depicted by box 146.

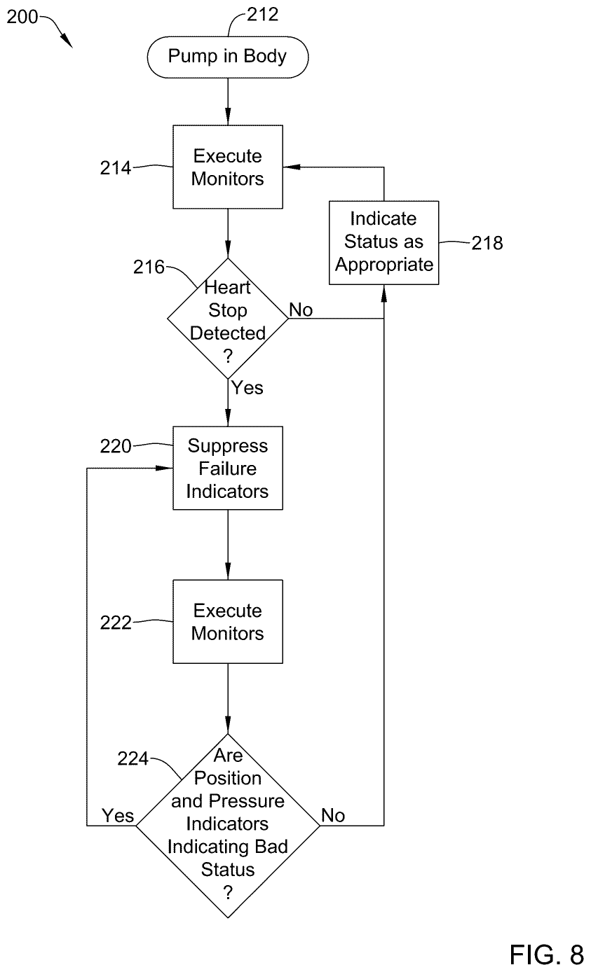

FIG. 8 is schematic diagram of an illustrative method 200 for providing automatic indications of a sudden stoppage of a patient's heart. FIG. 8 illustrates that a blood pump may be positioned in the heart of a patient 212. FIG. 8 further indicates that a motor torque sensor may be monitoring the position of the blood pump with the patient's heart, as depicted by box 214. Further, FIG. 8 further indicates that a blood pressure sensor may be monitoring the pressure within the patient's heart, as depicted by box 214. Based on comparing the motor toque sensed by the motor sensor to a pre-set threshold and the pressure in the patient's heart sensed by the pressure sensor to a pre-set threshold, the stoppage of the patient's heart may be detected 216. If the system determines the patient's heart has not stopped, the system may indicate that the blood pump is out of position, the pressure sensor has failed and/or the system is operating in a normal operating condition 218. However, if the system determines the patient's heart has stopped, the system may suppress 220 the out of position failure indicator and the pressure sensor failure indicator. Further, FIG. 8 indicates that after the system suppresses the out of position failure indicator and the pressure sensor failure, the motor torque sensor may monitor the position of the blood pump with the patient's heart and the pressure sensor may monitor the pressure within the patient's heart, as depicted by box 222. Further, FIG. 8 further indicates that if the position sensor determines the blood pump is out of position and the pressure sensor determines that the pressure or pressure rate in the heart is abnormally low, as depicted by box 224, then the system may indicate that the blood pump is out of position, the pressure sensor has failed and/or the system is operating in a normal operating condition 218.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A method for monitoring the heart, the method comprising:
advancing a cardiac pump adjacent to a left ventricle of the heart;
sensing a first parameter from a first sensor coupled to the cardiac pump;
sensing a second parameter from a second sensor coupled to the cardiac pump;
comparing the first parameter to a first threshold value;
comparing the second parameter to a second threshold value; and
increasing an output of blood out of the cardiac pump based on the comparison of the first parameter to the first threshold value and the comparison of the second parameter to the second threshold value.

2. The method of claim 1, wherein the first parameter is a torque of the cardiac pump.

3. The method of claim 2, wherein the second parameter is a blood pressure gradient in the aorta.

4. The method of claim 2, wherein the second parameter is a blood pressure gradient in the left ventricle.

5. The method of claim 1, wherein the first parameter is a pulsatility of a motor of the cardiac pump.

6. The method of claim 5, wherein the pulsatility corresponds to a signal selected from the group consisting of a current, a voltage, or a speed of the motor of the cardiac pump.

7. The method of claim 4, wherein the pulsatility corresponds to a position of the cardiac pump relative to the left ventricle.

8. The method of claim 1, wherein the first sensor is a torque sensor.

9. The method of claim 1, wherein the second sensor is a pressure sensor.

10. The method of claim 1, wherein the first parameter is compared to the first threshold value simultaneous with the second parameter being compared to the second threshold value.

11. The method of claim 1, wherein speed of the cardiac pump is increased for a pre-set period of time.

12. The method of claim 11, wherein the speed of the cardiac pump automatically decreases after the pre-set period of time expires.

13. The method of claim 1, further comprising generating an alarm coincident with increasing the speed of the cardiac pump.

14. The method of claim 1, wherein the second sensor is attached to an outer surface of the distal end region of the cardiac pump.

15. A method for monitoring the heart, the method comprising:
advancing a cardiac pump adjacent to a left ventricle of the heart;
sensing a first parameter from a first sensor coupled to the cardiac pump;
sensing a second parameter from a second sensor coupled to the cardiac pump comparing the first parameter to a first threshold value;
comparing the second parameter to a second threshold value; and
automatically adjusting a flow of blood out of the cardiac pump based on the comparison of the first parameter to the first threshold value and the comparison of the second parameter to the second threshold value.

16. The method of claim 15, wherein the first parameter is a torque of the cardiac pump.

17. The method of claim 16, wherein the second parameter is a blood pressure gradient.

18. The method of claim 15, wherein the first parameter is compared to the first threshold value simultaneous with the second parameter being compared to the second threshold value.

19. The method of claim 15, wherein speed of the cardiac pump is increased for a pre-set period of time.

20. A cardiac pump system, comprising:
a console including a processor; and
a cardiac pump in communication with the console, the cardiac pump including a motor, a first sensor and a second sensor;
wherein the first sensor is configured to sense a first parameter;
wherein the second sensor is configured to sense a second parameter; and
wherein the speed of the motor is configured to increase based on a comparison of the first parameter to a first threshold value and a comparison of a second parameter to a second threshold value.
